(19) United States
(12) Patent Application Publication
(10) Pub. No.: US 2023/0389815 A1
(43) Pub. Date: Dec. 7, 2023

(54) BLOOD FLOW MANAGEMENT DEVICE AND BLOOD FLOW MANAGEMENT SYSTEM

(71) Applicant: KYOCERA Corporation, Kyoto-shi, Kyoto (JP)

(72) Inventors: Ikutaro SAWADA, Kyoto-shi, Kyoto (JP); Yushi NAGASAKA, Kyoto-shi, Kyoto (JP); Taisei MANO, Kyoto-shi, Kyoto (JP)

(21) Appl. No.: 18/034,306

(22) PCT Filed: Dec. 6, 2021
(86) PCT No.: PCT/JP2021/044607

§ 371 Date: Apr. 27, 2023

(30) Foreign Application Priority Data
Dec. 17, 2020 (JP) 2020-209450

(51) Int. Cl. A61B 5/026; A61M 1/36

(52) U.S. Cl. CPC A61B 5/0261; A61M 1/3607; A61M 2205/3334

(57) ABSTRACT

A blood flow management device includes: a blood flow sensor that is capable of acquiring blood flow data being data related to blood flow by being arranged in direct or indirect contact with a measurement portion of a target site of a subject; and a blood flow adjustment means that adjusts the blood flow in the target site in accordance with the blood flow data.

### TECHNICAL FIELD

The present disclosure relates to a blood flow management device.

### BACKGROUND OF INVENTION

In a cancer medication therapy, for example, it is known that CIPN (chemotherapy-induced peripheral neuropathy) such as hypoesthesia and paresthesia in the distal portion of the extremities can be caused as a side effect of a taxane- or platinum-based anticancer agent.

As a therapeutic and preventive strategy for CIPN, reduction of blood flow in the distal portion of the extremities where the symptom of CIPN may occur is effective. As one example, a cooling therapy has been proposed. The cooling therapy locally cools the distal portion of the extremities, where the symptom of CIPN may occur, to reduce blood flow, thereby decreasing the pharmaceutical exposure in the distal portion and reducing the occurrence of CIPN.

In the cooling therapy described above, a commercially-available cold storage product such as a cold insulator, or a medical ice glove or a medical ice sock is conventionally used as a cooling means. Patent Document 1 discloses a damage suppression cooling device. The damage suppression cooling device controls a cooling capacity or a cooling temperature for each site (damage suppression site) that may be damaged by an anticancer agent or each blood flow system site (damage suppression target site) of the damage suppression site, and performs damage suppression cooling.

A compression therapy has been proposed as another example. The compression therapy involves compressing the distal portion of the extremities, where the symptom of CIPN may occur, to reduce blood flow, thereby decreasing the pharmaceutical exposure in the distal portion and reducing the occurrence of CIPN.

For example, the compression therapy described above uses a method of reducing blood flow in a hand by wearing two layers of elastic gloves having a size smaller than the size of a patient's hand on the patient's hand.

### CITATION LIST

### Patent Literature

Patent Document 1: JP 2019-98132 A

### SUMMARY

In an aspect of the present disclosure, a blood flow management device includes: a blood flow sensor that is arranged in direct or indirect contact with a measurement portion of a target site and can acquire blood flow data being data related to blood flow; and a blood flow adjustment means that adjusts the blood flow in the target site in accordance with the blood flow data.

### BRIEF DESCRIPTION OF THE DRAWINGS

FIG. 1 is a schematic view illustrating a cooling device according to a first embodiment of the present disclosure.

FIG. 2 is a cross-sectional view taken along line II-II indicated by arrows in FIG. 1.

FIG. 3 is a cross-sectional view taken along line III-III indicated by arrows in FIG. 1.

FIG. 4 is a functional block diagram illustrating a configuration example of a cooling management system according to the first embodiment of the present disclosure.

FIG. 5 is a flowchart illustrating an example of a flow of a cooling process by the cooling device described above.

FIG. 6 is a flowchart illustrating an example of a flow of a cooling process by the cooling device described above.

FIG. 7 is a cross-sectional view of a variation of a temperature adjustment device according to the first embodiment of the present disclosure.

FIG. 8 is a cross-sectional view of the variation described above.

FIG. 9 is a plan view of a variation of the temperature adjustment device according to the first embodiment of the present disclosure.

FIG. 10 is a cross-sectional view of the variation described above.

FIG. 11 is an enlarged view illustrating a configuration of a fingertip portion of the variation described above.

FIG. 12 is a plan view of a variation of the temperature adjustment device according to the first embodiment of the present disclosure.

FIG. 13 is a schematic view illustrating an outline of a pressure device according to the first embodiment of the present disclosure.

FIG. 14 is a cross-sectional view taken along line II-II indicated by arrows in FIG. 1.

FIG. 15 is a functional block diagram illustrating a configuration example of a pressure management system according to the first embodiment of the present disclosure.

FIG. 16 is a flowchart illustrating an example of a flow of a pressure management process by the pressure device illustrated in FIG. 3.

FIG. 17 is a functional block diagram illustrating a variation of a configuration of a pressure management system according to a second embodiment of the present disclosure.

FIG. 18 is a flowchart illustrating an example of a flow of a pressure management process by the pressure device illustrated in FIG. 5.

FIG. 19 is a diagram illustrating a power spectrum when a hand is not compressed.

FIG. 20 is a diagram illustrating a power spectrum when a hand is compressed.

### DESCRIPTION OF EMBODIMENTS

In the damage suppression cooling device disclosed in Patent Document 1, improvement in the accuracy of cooling temperature control is required to reduce the possibility of frostbite due to excessive cooling.

Known techniques for compression therapy may cause ischemic symptoms due to excessive compression, and management of blood flow in a compression site is required. According to a blood flow management device of the present disclosure, a blood flow management device with improved accuracy of measurement by a sensor can be provided. The blood flow management device of the present disclosure will be described in detail below.

The blood flow management device of the present disclosure is, for example, a device that can be used to perform blood flow management as a therapeutic and preventive strategy for CIPN which is a side effect of an anticancer agent. In other words, the blood flow management device of the present disclosure is a device that manages blood flow in a target site. In the present specification, the target site is, for example, a site where reduction of the possibility of CIPN symptoms is desired, and refers to, for example, the distal portion of the extremities, typically a hand (from a wrist to fingertips) or a foot (from an ankle to a toe). The target site is not limited to the exemplified portions and may include a lower arm or a lower thigh. A subject means a person in need of a blood flow management treatment, such as a patient.

An exemplary aspect of the blood flow management device of the present disclosure is a cooling device that changes blood flow, for example, by cooling a target site and manages the blood flow in the target site. Another exemplary aspect of the present disclosure is a compression device that changes blood flow by compressing a target site and, if necessary, the upstream of the target site (a side closer to the heart) and manages the blood flow in the target site. The cooling device will be described in detail in the first embodiment, and the compression device will be described in the second embodiment.

### First Embodiment

An embodiment of the present disclosure will be described in detail below.

### Cooling Device

A cooling device 500 of the present disclosure is a device that can be used to perform a cooling treatment as a therapeutic and preventive strategy for CIPN. In other words, the cooling device 500 of the present disclosure is the cooling device 500 that cools a cooling target site in need of cooling to perform the cooling treatment. The cooling target site is an example of the target site according to the present disclosure.

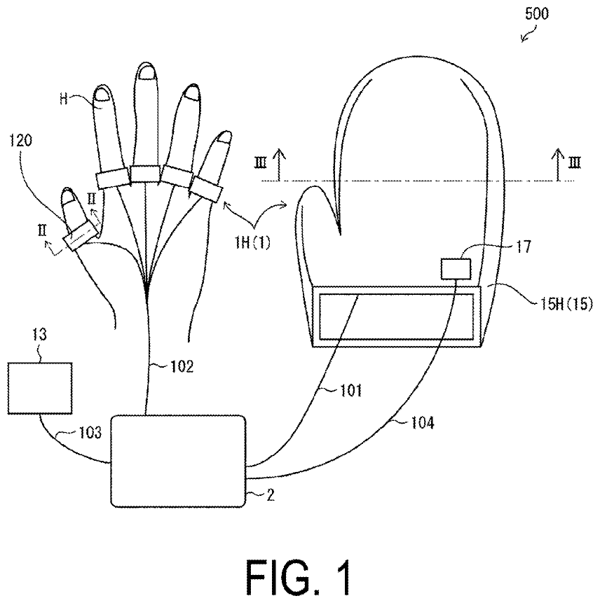

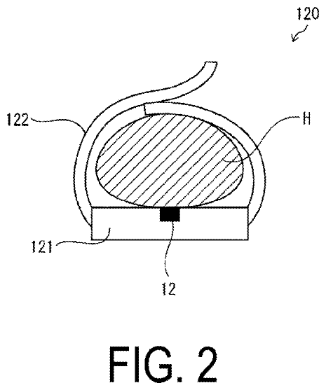

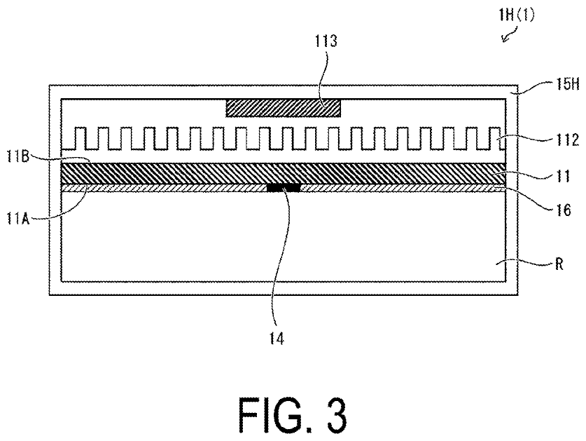

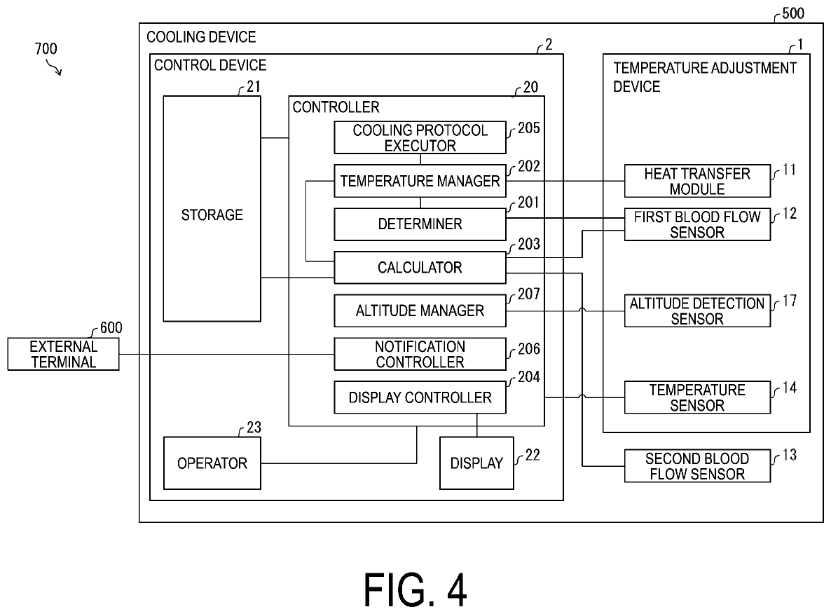

FIG. 1 is a schematic view illustrating an outline of the cooling device 500 according to the first embodiment. FIG. 2 is a cross-sectional view taken along line II-II indicated by arrows in FIG. 1. FIG. 3 is a cross-sectional view taken along line III-III indicated by arrows in FIG. 1. FIG. 4 is a functional block diagram illustrating a configuration example of a cooling management system 700.

As illustrated in FIG. 4, the cooling management system 700 includes the cooling device 500 and an external terminal 600 that makes a notification based on information from the cooling device 500. The cooling device 500 is an example of the blood flow management device according to the present disclosure. The cooling management system 700 is an example of the blood flow management system according to the present disclosure.

As illustrated in FIGS. 1 to 3, the cooling device 500 includes at least one temperature adjustment device 1, a control device 2, and a second blood flow sensor 13. The temperature adjustment device 1 includes a Peltier element 11 (heat transfer module), a first blood flow sensor 12, and an altitude detection sensor 17. More specifically, as illustrated in FIG. 1, the temperature adjustment device 1 includes an exterior casing 15, and the Peltier element 11 can be disposed inside the exterior casing 15 as illustrated in FIG. 3. The first blood flow sensor 12 can be detachably secured to a sensor fastener 120 as illustrated in FIG. 2. The altitude detection sensor 17 can be disposed at the exterior casing 15 as illustrated in FIG. 1. The Peltier element 11 can be connected to the control device 2 by a connection cable 101. The first blood flow sensor 12 can be connected to the control device 2 by a connection cable 102. The second blood flow sensor 13 can be connected to the control device 2 by a connection cable 103. The altitude detection sensor 17 can be connected to the control device 2 by a connection cable 104. The first blood flow sensor 12, the second blood flow sensor 13, and the altitude detection sensor 17 may be connected to the control device 2 by wire or wirelessly.

The temperature adjustment device 1 includes a cooling mechanism that is applied to each cooling target site and cools each cooling target site, and a monitoring mechanism that monitors a blood flow rate in each cooling target site. The cooling mechanism can be implemented by the Peltier element 11, and the monitoring mechanism can be implemented by the first blood flow sensor 12. As an example, FIG. 1 illustrates a temperature adjustment device 1H in which the right hand of a subject is the cooling target site. Although only the temperature adjustment device 1H is illustrated in FIG. 1, a plurality of temperature adjustment devices 1 may be connected to the control device 2. For example, when both hands are cooling target sites, two temperature adjustment devices 1 can be connected to the control device 2. When both hands and both feet are cooling target sites, four temperature adjustment devices 1 can be connected to the control device 2. Hereinafter, the configuration of the temperature adjustment device 1H will be described in detail. However, those skilled in the art can easily understand that another temperature adjustment device 1 in which a site other than a right hand is the cooling target site can have the same and/or similar configuration by appropriately changing the shape of the exterior casing 15.

The Peltier element 11 is an example of a heat transfer module that can adjust a surface temperature by controlling an applied voltage or current. The heat transfer module is an example of a blood flow adjustment means according to the present disclosure. That is, with the Peltier element 11, a cooling intensity can be adjusted (controlled) by controlling the applied voltage or current. By using the Peltier element 11 as a heat transfer module, the temperature can be quickly controlled. Hereinafter, for the sake of simplicity, a case where the voltage of the Peltier element 11 is controlled will be described, but the current of the Peltier element 11 may be controlled. As illustrated in the cross-sectional view of FIG. 3, the Peltier element 11 has a plate-like shape and includes a first surface 11A and a second surface 11B. The first surface 11A is a surface on an internal space R side of an exterior casing 15H into which a hand H of the subject can be inserted. In other words, the first surface 11A is a surface on the side facing a body surface. The second surface 11B is a surface on the side opposite from the first surface 11A. A heat sink 112 may be bonded to the second surface 11B. In the Peltier element 11, when a direct current flows to cool the first surface 11A, the second surface 11B generates heat. The heat sink 112 is a member that can dissipate the heat generated on the second surface 11B, and has a structure in which, for example, a large number of fins are provided on an aluminum alloy. The exterior casing 15 may include a fan 113 inside the exterior casing 15H so as to improve heat dissipation by circulating air warmed by the heat dissipated by the heat sink 112.

The shape, the size, and the number of the Peltier elements 11 disposed in the exterior casing 15H are not particularly limited. Any shape, any size, and any number of the Peltier elements 11 may be disposed in the exterior casing 15 depending on the shape of a cooling target site and/or the shape of the exterior casing 15. FIG. 3 illustrates an example in which the Peltier element 11 is disposed only on one side of the space R in the temperature adjustment device 1H, but the Peltier element 11 may be disposed on both sides of the space R. The positional relationship between the Peltier element 11 and the first blood flow sensor 12 is not particularly limited. For example, the Peltier element 11 may be disposed so as to overlap the first blood flow sensor 12 in a plane perspective when the hand H wearing the first blood flow sensor 12 is inserted into the exterior casing 15H, and the exterior casing 15H is viewed from the back side of the hand H. The Peltier element 11 and the first blood flow sensor 12 may be spaced apart from each other in a plane perspective when the hand H wearing the first blood flow sensor 12 is inserted into the exterior casing 15H, and the exterior casing 15H is viewed from the back side of the hand H.

Disposing the Peltier element 11 and the first blood flow sensor 12 apart from each other can reduce the influence of temperature changes caused by the Peltier element 11 on performance such as circuit characteristics of the first blood flow sensor 12. Thus, the accuracy of measurement by the first blood flow sensor 12 can be further improved.

The temperature adjustment device 1 may include a cushion member 16 between the Peltier element 11 and a cooling target site. The cushion member 16 reduces the possibility of a skin damage due to direct contact of the cooling target site with the first surface 11A of the Peltier element 11 when the temperature of the first surface 11A decreases. The cushion member 16 may be formed so as to cover the entire cooling target site. The cushion member 16 may be made of any material that can reduce excessive stimulation caused by a direct contact with the first surface 11A. The cushion member 16 is composed of, for example, a non-woven fabric, a stretchable knitted fabric, or a polyethylene sheet. The non-woven fabric is, for example, a medical non-woven fabric obtained by a production method such as a spun-lace method, a melt-blown method, an SMS (Spunbonded-Meltblown-Spunbond) method, or a flash-spun method. The stretchable knitted fabric is, for example, a knitted fabric having stretchability formed by using elastic fibers, stretchable fibers, or the like. FIG. 3 illustrates an exemplary configuration of the cushion member 16. As illustrated in FIG. 3, the exterior casing 15 may include the cushion member 16 between the Peltier element 11 and the region R. The configuration of the cushion member 16 is not limited to the example illustrated in FIG. 3, and may be such that, for example, when the cooling target site is a hand, the hand wearing the first blood flow sensor 12 is covered with the cushion member 16 having a glove (or mitten) shape and inserted into the exterior casing 15.

When the temperature adjustment device 1 includes the cushion member 16, the cooling by the Peltier element 11 can be spread to the entire cooling target site. Thus, the entire cooling target site can be cooled. The possibility of occurrence of frostbite due to local cooling can be reduced.

As illustrated in FIG. 3, the temperature adjustment device 1 may include a temperature sensor 14 that measures the temperature of the first surface 11A of the Peltier element 11. The temperature sensor 14 can be disposed at a position adjacent to the Peltier element 11 and can be connected to the control device 2 by wire or wirelessly. The temperature sensor 14 may be built in the Peltier element 11. That is, for example, the temperature sensor 14 may be disposed between rows of thermoelectric elements composed of a plurality of thermoelectric elements in the first surface 11A of the Peltier element 11. The temperature of the Peltier element 11 can be controlled by controlling an applied voltage. When the temperature sensor 14 is provided, the temperature of the first surface 11A of the Peltier element 11 can be grasped. By grasping the actual temperature of the first surface 11A using the temperature sensor 14, the temperature can be controlled more appropriately. The surface temperature of the first surface 11A measured by the temperature sensor 14 can be displayed on a display 22 to be described later, for example.

The exterior casing 15H is an example of the exterior casing 15, and is a member having a space capable of accommodating therein at least a cooling target site, the Peltier element 11, and the first blood flow sensor 12. FIG. 1 illustrates, as an example, the exterior casing 15H in a case where the cooling target site is a hand. The exterior casing 15H has a mitten shape into which the hand H of a patient can be inserted. The exterior casing 15H is formed by use of a material that is unlikely to be deformed or transformed by the temperature change of the Peltier element 11. In order to improve the cooling efficiency of the Peltier element 11, the material of the exterior casing 15H may be a heat-insulating material. More specifically, the exterior casing 15H can be formed by use of, for example, an inner cotton fabric or a urethane material.

The exterior casing 15H is an example of the exterior casing 15, and the exterior casing 15 can have an appropriate shape depending on the shape of the cooling target site. For example, when the cooling target site is a hand, the exterior casing 15 may be a glove type having a shape branched into respective finger portions. When the cooling target site is a foot, the exterior casing 15 may have, for example, a sock shape.

The first blood flow sensor 12 is a sensor that can acquire blood flow data at a measurement portion (first measurement portion) of the cooling target site. The data related to blood flow acquired by the first blood flow sensor 12 is referred to as first blood flow data. The first blood flow sensor 12 may be, for example, a flow rate calculation device using the Doppler effect of light. For example, such a flow rate calculation device includes a light emitting element, a light receiving element, and a flow rate calculator. The light emitting element emits light having a wavelength of 600 to 900 nm to a measurement object. The light receiving element receives overall light scattered by a substance including the measurement object among the light emitted from the light emitting element. The flow rate calculator calculates the flow rate of a fluid based on the frequency component of the scattered light received by the light receiving element, the total power of received light signals, and a proportional constant K. Here, the total power of received light signals means a value of a light intensity I².

More specifically, since the frequency of the scattered light scattered by the fluid shifts (Doppler shift) due to the Doppler effect proportional to the movement speed of the fluid, the flow rate can be measured using the frequency shift. That is, the flow rate calculation device described above can acquire a beat signal generated by the interference of light scattered from a stationary substance with light scattered from a moving substance, by using the Doppler effect. The beat signal indicates a relationship between intensity and time, and a power spectrum indicating a relationship between power and frequency can be acquired by performing Fourier transform. That is, the flow rate calculation device described above can also be said to include a power spectrum generator that derives a power spectrum indicating a relationship between power and frequency based on the frequency component of the scattered light. Since a power value of a power spectrum varies depending on a flow rate of a fluid, the first order moment of the power spectrum is a value equivalent to the flow rate. The first order moment can be obtained by the following integral equation (1).

Equation 1

Q=∫ₐ^{b}fPdf (1)

Since an actually obtained output value is discrete, calculation is performed based on the following equation (2) as an operation equivalent to the integration (equation 1).

$$Q = {\sum }_{a}^{b} 1 P \qquad ( 2 )$$

In the above equations (1) and (2), Q represents a value equivalent to the flow rate, f represents a frequency, and P represents a power. Further, a and b respectively represent the lower limit and the upper limit of the frequency f used in the operation.

By using the flow rate calculation device described above, the blood flow rate (first blood flow rate) in the first measurement portion can be calculated based on the frequency component (first blood flow data) of the scattered light acquired at the first measurement portion, the total power of received light signals, and the proportional constant K.

As illustrated in FIG. 2, the first blood flow sensor 12 is fixed to the first measurement portion by the sensor fastener 120. The sensor fastener 120 is a member that fixes the first blood flow sensor 12 with respect to the first measurement portion. The sensor fastener 120 includes a sensor support member 121 to which the first blood flow sensor 12 is attached, and a band 122 for fixing the first blood flow sensor and the sensor support member 121 to the first measurement portion. The band 122 is a band-shaped member that can fix the first blood flow sensor 12 so as to fit the shape of the first measurement portion, and can be formed by using, for example, a hook-and-loop fastener such as a magic tape (trade name). The sensor fastener 120 is not limited to the configuration described above, and may have any configuration as long as the first blood flow sensor 12 can be fixed with respect to the first measurement portion. By providing the sensor fastener 120, the first blood flow sensor 12 can reduce the possibility of displacement of the first blood flow sensor 12 from the first measurement portion. Thus, the accuracy of measurement by the sensor can be further improved. A plate member having translucency and low thermal conductivity may be disposed between the first blood flow sensor 12 and the first measurement portion. This can reduce the possibility of adhesion of the first blood flow sensor 12 to the measurement portion during cooling.

The first measurement portion can be arbitrarily set, and is typically set at a position where confirmation of the reduction in blood flow by the cooling treatment is desired. For example, in order to confirm the reduction in blood flow in a digit of a hand or a foot, the first measurement portion may be set at a base or a tip of the digit. The first blood flow sensor 12 can be arranged in contact with a skin surface of the first measurement portion. The first blood flow sensor 12 may be arranged on a palm when the cooling target site is a hand, or may be arranged on a sole when the cooling target site is a foot. When the first blood flow sensor 12 is arranged on the palm or the sole, a contact surface with which the first blood flow sensor 12 comes into contact is soft, and thus the adherence of the first blood flow sensor 12 is improved. Since a palm side or a sole side is a portion where peripheral blood vessel distribution is developed, blood flow data can be relatively easily acquired by the first blood flow sensor 12 as compared to the back side of a hand or the top side of a foot.

The first blood flow sensor 12 may be arranged at a side surface of a digit of a hand or a foot. By placing the first blood flow sensor 12 at the side surface of the digit, blood flow data can be acquired by the first blood flow sensor 12 even when the Peltier element 11 is arranged on both sides of the hand or the foot. Since an artery passes near a skin at the side surface of the digit, blood flow data can be relatively easily acquired by the first blood flow sensor 12.

The first blood flow sensor 12 may be arranged at a digit of a hand or a foot on the same surface as a nail (surface on the back side of the hand or the top side of the foot), or at the back of the hand or the top of the foot. By placing the first blood flow sensor 12 at the back of a hand or the top of the foot, the Peltier element 11 can be arranged over the entire surface of the palm side or the sole side. Since the palm side or the sole side is softer than the back side of the hand or the top side of the foot, the cooling surface area becomes large when the palm side or the sole side is brought into contact with the Peltier element 11, and the palm side or the sole side can be efficiently cooled.

A plurality of first blood flow sensors 12 may be arranged for one cooling target site (for example, a right hand, a left hand, a right foot, or a left foot). In FIG. 1, the first blood flow sensor 12 is fixed to each of five fingers. However, the position and the number of the first blood flow sensors 12 for one cooling target site are not limited to the example illustrated in FIG. 1.

The second blood flow sensor 13 is a sensor that is arranged at a position (second measurement portion) different from the position of the first blood flow sensor 12 on the subject and can acquire blood flow data at the second measurement site. The second blood flow sensor 13 can be a sensor having a configuration same as and/or similar to the configuration of the first blood flow sensor 12. The blood flow data acquired by the second blood flow sensor 13 is referred to as second blood flow data. A second blood flow rate obtained from the second blood flow data may be used as a reference value for the first blood flow rate. A problem with the blood flow sensor is that the blood flow sensor may have a difficulty, due to the principle thereof, in making a determination based on absolute values. When the second blood flow rate serving as a reference value is used and a change in the first blood flow rate due to cooling is identified as a change in a relative value with reference to the second blood flow rate, a determination criterion such as an absolute threshold value can be set. That is, by providing the second blood flow sensor 13, changes in the blood flow rate due to cooling can be grasped more accurately. Thus, more appropriate temperature management can be performed.

The second measurement portion can be arbitrarily set. As described above, in order to make a determination using a relative value, the second measurement portion can be set at a site that is separated from the first measurement portion and is closer to the heart of the subject than the first measurement portion. For example, the second measurement portion can be a wrist or an upper arm of the subject. The second blood flow sensor 13 can be arranged in contact with a skin surface at the second measurement portion and can be fixed to the second measurement portion by a sensor fastener (not illustrated).

The altitude detection sensor 17 is a sensor that can acquire altitude data related to the altitude of the cooling target site. In the measurement of blood flow, a change in the positional relationship between the measurement portion (cooling target site) and the heart may affect the measurement result. By providing the altitude detection sensor 17, a change in the positional relationship between the measurement portion and the heart can be grasped. As the altitude detection sensor 17, for example, an acceleration sensor and/or an air pressure sensor can be used. That is, the altitude data acquired by the altitude detection sensor 17 is, for example, acceleration information obtained from the acceleration sensor and/or air pressure information obtained from the air pressure sensor. The altitude detection sensor 17 can transmit the altitude data to an altitude manager 207 to be described later. Either the acceleration sensor or the air pressure sensor may be used as the altitude detection sensor 17. However, by using the acceleration sensor and the air pressure sensor in combination, the altitude manager 207 can calculate a displacement in altitude more accurately.

The altitude detection sensor 17 may be attached to the temperature adjustment device 1, for example. For example, as illustrated in FIG. 1, the altitude detection sensor 17 may be detachably attached to an outer surface of the exterior casing 15. The mounting position of the altitude detection sensor 17 is not limited to the example of FIG. 1. The attachment position of the altitude detection sensor 17 may be any position as long as the altitude detection sensor 17 can move in conjunction with the cooling target site when the cooling target site moves with the movement of the body. For example, when the cooling target site is a hand, the altitude detection sensor 17 may be fixed to a part of the hand, a wrist, or the like by a sensor fastener (not illustrated) that can have a configuration same as and/or similar to the configuration of the sensor fastener 120.

The external terminal 600 is a terminal that can make a notification to prompt the user to make a confirmation based on information from the cooling device 500. In the present specification, the user means a person who operates the cooling device 500, and is, for example, a medical doctor or a nurse who performs the cooling treatment on the subject. During the cooling treatment performed by the cooling device 500, the user has difficulty in constantly monitoring the cooling by the cooling device 500. The external terminal 600 is communicably connected to the control device 2 by wire or wirelessly, and can notify the user at a remote location (for example, a nurse station or the like) of information from the cooling device 500 by display, sound, vibration, lamp lighting, or the like. By providing the external terminal 600, temperature management for the cooling therapy can be performed while human resources can be reduced.

### Control Device

The control device 2 will be described using FIG. 4. The control device 2 includes a controller 20, a storage 21, a display 22, and an operator 23. The controller 20 includes a determiner 201, a temperature manager 202, a calculator 203, a display controller 204, a cooling protocol executor 205, a notification controller 206, and the altitude manager 207.

The controller 20 is composed of a CPU (Central Processing Unit) or the like, can execute a program stored in the storage 21, for example, and can perform overall control on each component of the cooling device 500. The storage 21 is composed of a non-volatile storage medium such as a hard disk or a flash memory, and can store various kinds of information supplied from the controller 20. The information stored in the storage 21 can be read out by the controller 20 as appropriate. The display 22 is a type of display, and can display various types of information on a display surface thereof based on an instruction from the display controller 204. The operator 23 is a component that receives an input operation of the user, and may be composed of a switch, a button, a touch screen, or the like.

The determiner 201 can determine whether or not the first blood flow rate obtained from the first blood flow data acquired by the first blood flow sensor 12 is within an appropriate range. The appropriate range is an appropriate range of a blood flow rate arbitrarily defined depending on the cooling target site. When a plurality of first blood flow sensors 12 are arranged at the cooling target site, the determiner 201 may determine whether or not a representative first blood flow rate calculated based on a plurality of pieces first blood flow data acquired from the plurality of first blood flow sensors 12 is within the appropriate range. The representative first blood flow rate obtained from the plurality of pieces of first blood flow data may be calculated by the calculator 203 to be described later. The representative first blood flow rate may be, for example, an average value of a plurality of first blood flow rates obtained from the plurality of pieces of first blood flow data or a median value of the plurality of first blood flow rates. The determiner 201 may determine whether or not information (first numerical data) correlated with the first blood flow rate obtained from the first blood flow data is within a prescribed appropriate range. The appropriate range may be set in advance in accordance with the type of information indicated by the first numerical data.

The temperature manager 202 can control the Peltier element 11 based on the determination result of the determiner 201. More specifically, the temperature manager 202 can control the voltage applied to the Peltier element 11 based on the determination result of the determiner 201.

The calculator 203 can calculate a blood flow change amount at the cooling target site based on the first blood flow data and the second blood flow data. For example, the calculator 203 can calculate the blood flow change amount based on the first blood flow rate obtained from the first blood flow data and the second blood flow rate obtained from the second blood flow data. The calculator 203 may calculate the blood flow change amount based on the first numerical data described above and information that is obtained from the second blood flow data and correlated with the second blood flow rate (second numerical data). The calculator 203 may calculate the blood flow change amount based on the first blood flow rate and an initial first blood flow rate before the start of the cooling process. That is, the controller 20 may include the calculator 203 that calculates the blood flow change amount at the cooling target site based on the first blood flow rate and the initial first blood flow rate before the start of the cooling process. As in this configuration, when the initial first blood flow rate before the start of the cooling process is used as a reference value and a change in the first blood flow rate due to cooling is identified as a change in a relative value with reference to the initial first blood flow rate before the start of the cooling process, a determination criterion such as an absolute threshold value can be set. That is, changes in the blood flow rate due to cooling can be grasped more accurately. Thus, more appropriate temperature management can be performed.

The display controller 204 controls information displayed on the display 22. For example, the display controller 204 can output temperature data received from the temperature sensor 14 to the display 22, thereby causing the display 22 to display the temperature data. The cooling protocol executor 205 can control a voltage applied to the Peltier element 11 in accordance with a preset cooling protocol. The notification controller 206 can drive the external terminal 600 in accordance with information supplied from the controller 20 to notify the user of the information.

The altitude manager 207 receives altitude data from the altitude detection sensor 17 and, based on the altitude data, calculates a displacement of the altitude of the cooling target site with respect to a cooling start time. When the displacement of the altitude of the cooling target site with respect to the cooling start time becomes equal to or greater than a predetermined value, the altitude manager 207 may transmit, to the display controller 204, altitude warning information notifying that a displacement equal to or greater than the predetermined value has occurred. The display controller 204 can output the altitude warning information to the display 22, thereby causing the display 22 to display that a displacement equal to or greater than the predetermined value has occurred.

When a displacement exceeding a predetermined displacement amount occurs, the altitude manager 207 may transmit, to the temperature manager 202, altitude warning information indicating that a displacement exceeding a predetermined displacement amount has occurred. Upon receipt of the altitude warning information, the temperature manager 202 may temporarily stop the control flow of the voltage applied to the Peltier element 11. In other words, upon receipt of the altitude warning information, the temperature manager 202 maintains the voltage applied to the Peltier element 11 at the time when the altitude warning information is received, and the controller 20 does not need to perform the next processing. When the displacement of the altitude of the cooling target site obtained from the altitude detection sensor 17 returns to the predetermined displacement amount or less, the altitude manager 207 may transmit altitude warning cancellation information to the temperature manager 202. Upon receipt of the altitude warning cancellation information, the temperature manager 202 may resume the control flow of the voltage applied to the Peltier element 11.

Further, the altitude manager 207 may correct the first blood flow rate using the calculated displacement of the altitude. For example, the altitude manager 207 calculates a corrected first blood flow rate using the following equation (1).

Corrected first blood flow rate=first blood flow rate×(1β×altitude displacement amount) (1)

In the above equation (1), β is an arbitrary correction coefficient. The determiner 201 may determine whether or not the first blood flow rate is within an appropriate range by using the corrected first blood flow rate that can be acquired from the altitude manager 207 as the first blood flow rate.

### Cooling Process 1

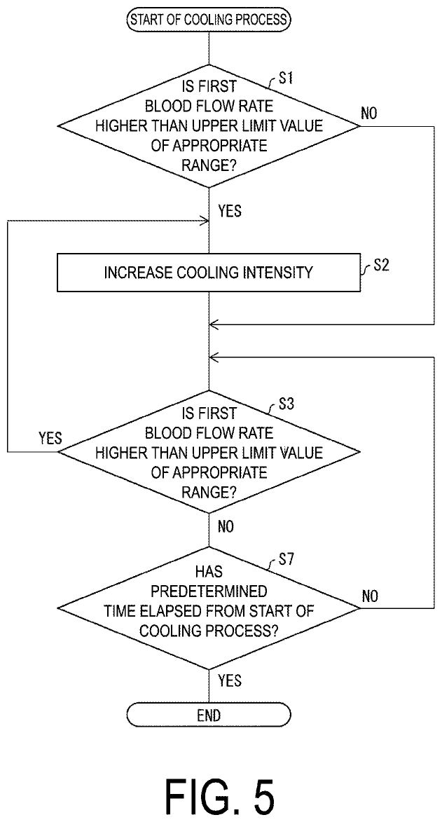

An example of the cooling process by the controller 20 will be described using FIG. 5. FIG. 5 is a flowchart illustrating an example of a flow of the cooling process by the cooling device 500 according to the first embodiment.

As illustrated in FIG. 5, when the cooling device 500 starts the cooling process, the determiner 201 acquires the first blood flow rate from the first blood flow sensor 12 and determines whether or not the acquired first blood flow rate is higher than the upper limit value of the appropriate range (S1). The upper limit value of the appropriate range may be arbitrarily set. For example, a value of the blood flow rate at which the pharmaceutical exposure in the cooling target site is determined to be reducible can be used as the upper limit value of the appropriate range. When the determiner 201 determines that the first blood flow rate is higher than the upper limit value of the appropriate range (S1: YES), the temperature manager 202 controls the voltage applied to the Peltier element 11 to increase the cooling intensity (S2). When the determiner 201 determines that the first blood flow rate is not higher than (is equal to or lower than) the upper limit value of the appropriate range (S1: YES), the cooling intensity is maintained, and the processing proceeds to the next determination (S3).

The determiner 201 acquires again the first blood flow rate from the first blood flow sensor 12 and determines whether or not the acquired first blood flow rate is higher than the upper limit value of the appropriate range (S3). When the determiner 201 determines that the first blood flow rate is higher than the upper limit value of the appropriate range (S3: YES), the temperature manager 202 controls the voltage applied to the Peltier element 11 to increase the cooling intensity (returns to the processing in S2). When the determiner 201 determines that the first blood flow rate is not higher than the upper limit value of the appropriate range (S3: NO), the cooling intensity is maintained, and the processing proceeds to the next determination (S7).

The controller 20 determines whether or not a predetermined time has elapsed from the start of the cooling process (S7). The predetermined time is, for example, a time that can be arbitrarily set based on the time required for the administration of an anticancer agent. The predetermined time may be stored in the storage 21 as required cooling time information, for example. The predetermined time may be input from the operator 23 by the user. When the controller 20 determines that the predetermined time has elapsed (S7: YES), the controller 20 ends the cooling process. When the controller 20 determines that the predetermined time has not elapsed (S7: NO), the controller 20 returns to the processing in S3.

By the controller 20 performing the cooling process 1, temperature management can be performed such that the blood flow rate in the first measurement portion does not become higher than the upper limit value of the appropriate range of the blood flow rate. Consequently, the pharmaceutical exposure in the cooling target site can be reduced, thereby facilitating the management of the cooling therapy that reduces the occurrence of CIPN.

### Cooling Process 2

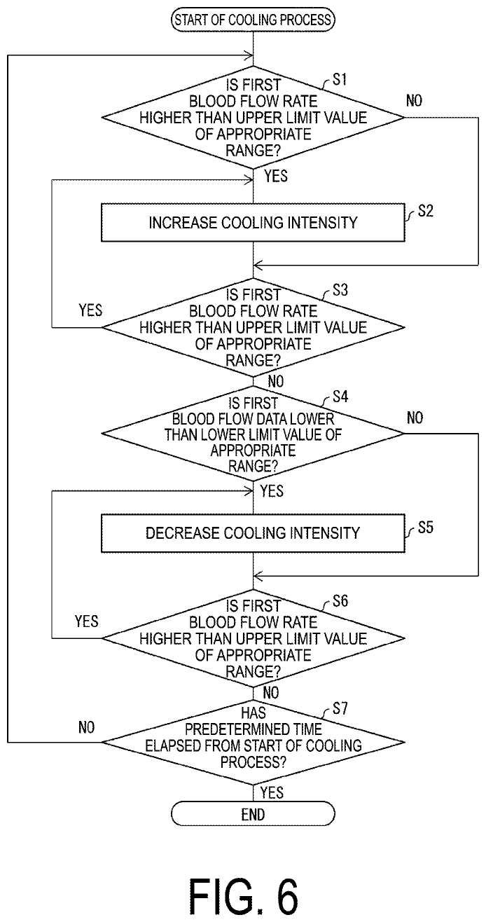

Another example of the cooling process by the controller 20 will be described using FIG. 6. FIG. 6 is a flowchart illustrating an example of a flow of the cooling process by the cooling device 500 according to the first embodiment. The cooling process 2 is different from the cooling process 1 in that the control is performed also in consideration of the lower limit value of the appropriate range. In the cooling process 1 and the cooling process 2, the same and/or similar processing is performed in the steps to which the same step numbers are assigned.

As illustrated in FIG. 6, when the cooling device 500 starts the cooling process, the determiner 201 executes the same and/or similar processing as in the above-described cooling process 1 from step S1 to step S3.

In step S3, when the determiner 201 determines that the first blood flow rate is higher than the upper limit value of the appropriate range (S3: NO), then the determiner 201 determines whether or not the first blood flow rate is lower than the lower limit value of the appropriate range (S4). The lower limit value of the appropriate range may be arbitrarily set. For example, a minimum blood flow rate for reducing the possibility of the occurrence of frostbite in the cooling target site can be used as the lower limit value of the appropriate range.

In step S4, when the determiner 201 determines that the first blood flow rate is lower than the lower limit value of the appropriate range (S4: YES), the temperature manager 202 controls the voltage applied to the Peltier element 11 to decrease the cooling intensity (S5). When the determiner 201 determines that the first blood flow rate is not lower than (is equal to or higher than) the lower limit value of the appropriate range (S4: NO), the cooling intensity is maintained, and the processing proceeds to the next determination (S6).

The determiner 201 acquires again the first blood flow rate from the first blood flow sensor 12 and determines whether or not the acquired first blood flow rate is lower than the lower limit value of the appropriate range (S6). When the determiner 201 determines that the first blood flow rate is lower than the lower limit value of the appropriate range (S6: YES), the temperature manager 202 controls the voltage applied to the Peltier element 11 to decrease the cooling intensity (returns to the processing in S5). When the determiner 201 determines that the first blood flow rate does not exceed the lower limit value of the appropriate range (S6: NO), the cooling intensity is maintained, and the processing proceeds to the next determination (S7).

When the controller 20 determines that a predetermined time has elapsed from the start of the cooling process (S7: YES), the controller 20 ends the cooling process. When the controller 20 determines that the predetermined time has not elapsed (S7: NO), the controller 20 returns to the processing in S1.

By the controller 20 performing the cooling process 2, temperature management can be performed such that the blood flow rate in the first measurement portion falls within the appropriate range of the blood flow rate. Consequently, the pharmaceutical exposure in the cooling target site can be reduced, and the management of the cooling therapy that can reduce the possibility of the occurrence of frostbite at the cooling target site can be facilitated.

### Another Control

When a series of the cooling processes is completed, the notification controller 206 may notify that the series of cooling processes has been completed via the external terminal 600. When an error occurs in the cooling device 500, the notification controller 206 may notify that the error has occurred in the cooling device 500 via the external terminal 600.

### Control According to Cooling Protocol

The controller 20 may include the cooling protocol executor 205 that controls the voltage applied to the Peltier element 11 in accordance with a preset cooling protocol. The cooling protocol can be created by the user depending on the type of a medical agent to be administered, an administration time of the medical agent, or the like. The cooling protocol may be stored in the storage 21 in advance. The user may input information necessary for creating the cooling protocol via the operator 23, and the information may be stored in the storage as the cooling protocol. The cooling protocol may be stored in a cloud server (not illustrated) that can be connected to the controller 20 via a communication network. The cooling protocol executor 205 can acquire the cooling protocol from the storage 21 or the cloud server. The cooling protocol executor 205 can execute, in accordance with the cooling protocol, at least one of cooling for an arbitrary predetermined time before and after the administration of the medical agent, cooling for an arbitrary predetermined time with consideration of the administration time of the medical agent, and warming after cooling for a predetermined time.

By providing the cooling protocol executor 205, a cooling treatment can be executed based on a desired cooling protocol in consideration of the type of a medical agent to be administered, the administration time of the medical agent, and the like.

With the above-described configuration, the cooling device 500 can more accurately measure the blood flow rate in the first measurement portion. By appropriately controlling the cooling while measuring the blood flow rate, the reduction in pharmaceutical exposure in the distal portion of the extremities and the reduction in the possibility of the occurrence of frostbite due to overcooling can be achieved in a compatible manner.

### First Variation

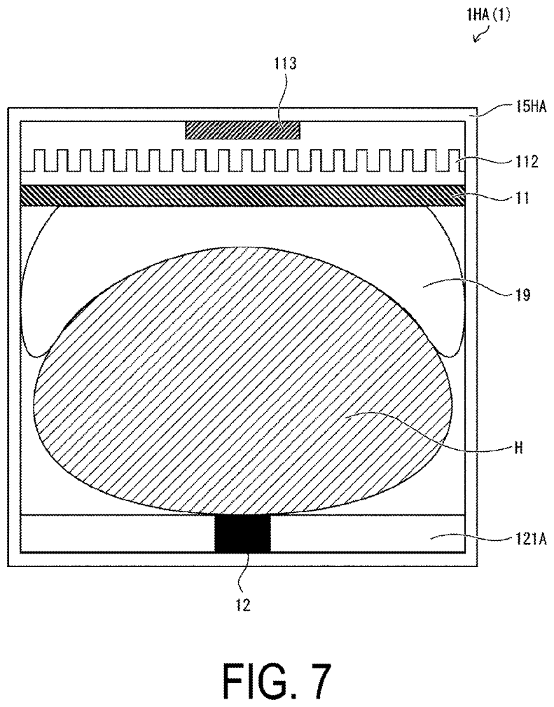

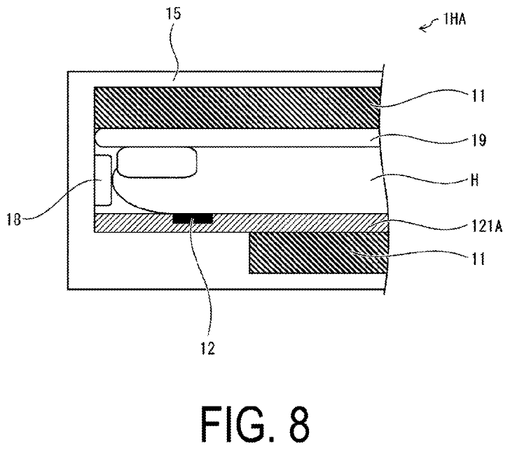

In a first variation, a variation of the temperature adjustment device 1H according to the first embodiment will be described with reference to FIGS. 7 and 8.

FIG. 7 is a cross-sectional view of an exemplary temperature adjustment device 1HA as a variation of the temperature adjustment device 1H. The cross-sectional view illustrated in FIG. 7 is a cross-sectional view obtained by cutting the temperature adjustment device 1HA along a plane perpendicular to a palm and perpendicular to an extending direction of a middle finger. The temperature adjustment device 1HA is different from the temperature adjustment device 1H described above in that the first blood flow sensor 12 is disposed in an exterior casing 15HA, that a pressure bag 19 is provided as at least a part of a pressurization mechanism, and that a switch 18 is provided. Members having the same functions as the members described in the embodiment described above are denoted by the same reference signs, and descriptions thereof will not be repeated. The same applies to other variations.

As illustrated in FIG. 7, the first blood flow sensor 12 may be disposed in the exterior casing 15HA. At this time, the first blood flow sensor 12 may be supported by a sensor support member 121A so that the position of the first blood flow sensor 12 does not shift in the exterior casing 15HA.

The pressurization mechanism can be configured to press a measurement portion against the first blood flow sensor 12 and/or press the first blood flow sensor 12 against the measurement portion in a state where the first blood flow sensor 12 and the measurement portion are in contact with each other.

For example, as illustrated in FIG. 7, the pressurization mechanism may include the pressure bag 19 expandable by a fluid that can be supplied from the outside. The pressure bag 19 can be disposed between the Peltier element 11 and a hand H as the cooling target site. At this time, the first blood flow sensor 12 can be disposed at a position on a side opposite to the pressure bag 19 across the hand H. The pressurization mechanism may include supply means for supplying a fluid to the pressure bag 19 and discharge means for discharging the fluid from the pressure bag 19. The fluid is not particularly limited, and a gas such as air or a liquid such as water can be used. The supply means is, for example, a pump when the fluid in the pressure bag 19 is a liquid, and is, for example, a valve when the fluid is a gas.

When the pressurization mechanism is provided, the controller 20 may include a pressurization mechanism controller (not illustrated). For example, the pressurization mechanism controller can start supplying the fluid to the pressure bag 19 by controlling the supply means when the cooling target site is determined to have been inserted to a specified position. The determination of insertion to the specified position will be described later. The pressurization mechanism controller can keep the internal pressure of the pressure bag 19 constant during the cooling treatment by controlling the supply means and/or the discharge means.

FIG. 8 is a cross-sectional view of the temperature adjustment device 1HA. The cross-sectional view illustrated in FIG. 8 is an enlarged cross-sectional view of a fingertip portion obtained by cutting the temperature adjustment device 1HA along a plane perpendicular to a palm and perpendicular to an extending direction of a middle finger.

FIG. 8 illustrates an example in which the Peltier element 11 is disposed on both sides (the palm side and the back side of the hand) of the hand (measurement portion). The Peltier element 11 disposed on the back side of the hand is arranged on a side opposite to the first blood flow sensor 12 across the hand. The Peltier element 11 disposed on the palm side is arranged at a position spaced apart from the first blood flow sensor 12 that is also disposed on the palm side.

As illustrated in FIG. 8, the temperature adjustment device 1HA may include the switch 18 on an inner wall of a distal portion of the exterior casing 15. When the temperature adjustment device 1HA includes the switch 18, the controller 20 may include a positioning determiner (not illustrated). For example, when the switch 18 is pushed by the cooling target site, the positioning determiner can determine that the cooling target site has been inserted to the specified position.

When the temperature adjustment device has the configuration of the temperature adjustment device 1HA, the controller 20 may start the cooling process 1 or the cooling process 2 according to the flowchart illustrated in FIG. 5 or 6 after the pressurization mechanism controller starts the supply of the fluid to the pressure bag 19.

After the cooling process 1 or the cooling process 2 is completed, the pressurization mechanism controller can reduce the internal pressure of the pressure bag 19 by controlling the supply means and/or the discharge means.

When the temperature adjustment device 1HA includes the pressurization mechanism, the first blood flow sensor 12 can be pressed against the first measurement portion at a constant contact pressure. Thus, the first blood flow rate can be obtained more accurately.

### Second Variation

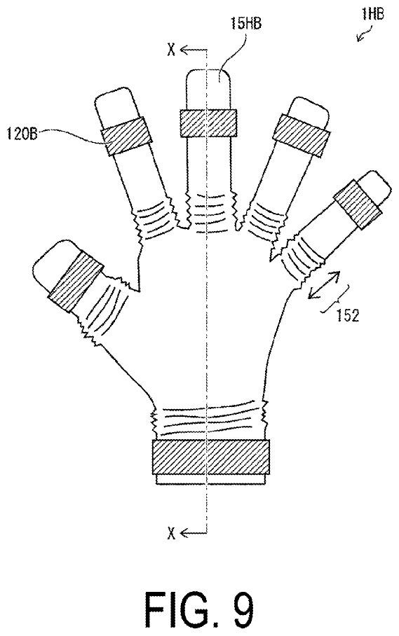

In a second variation, another variation of the temperature adjustment device 1H according to the first embodiment will be described with reference to FIGS. 9 to 11.

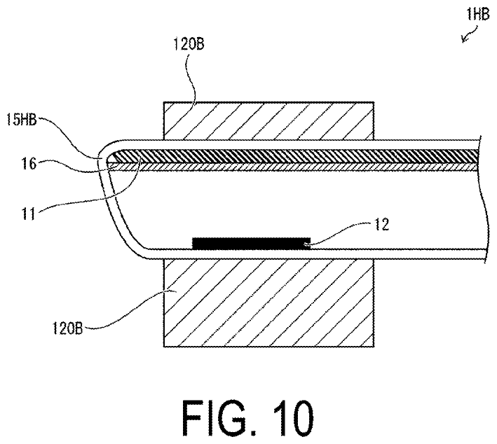

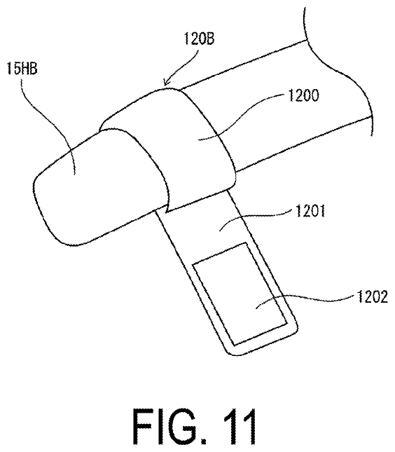

FIG. 9 is a plan view of an exemplary temperature adjustment device 1HB as a variation of the temperature adjustment device 1H. The plan view illustrated in FIG. 9 is a plan view in which the temperature adjustment device 1HB whose cooling target site is the right hand of a subject is viewed from the back side of the hand. FIG. 10 is a cross-sectional view of the temperature adjustment device 1HB. The cross-sectional view illustrated in FIG. 10 is an enlarged view of a fingertip portion in the cross-sectional view taken along line X-X indicated by arrows in FIG. 9. FIG. 11 is an enlarged view illustrating a configuration of the fingertip portion of the temperature adjustment device 1HB.

In the second variation, the temperature adjustment device 1HB includes an exterior casing 15HB, and the Peltier element 11 can be fixed inside the exterior casing 15HB as illustrated in FIG. 10. The first blood flow sensor 12 can be fixed to an inner surface of the exterior casing 15HB by any adhesion means at a position facing the pad of each finger when the hand H is inserted into the exterior casing 15HB. Although not illustrated, the exterior casing 15HB may include the altitude detection sensor 17, the heat sink 112, and/or a fan 113.

The exterior casing 15HB is a configuration example in which the cooling target site is the right hand and the first measurement portions are the tips of the fingers. The exterior casing 15HB has a glove shape having a five-branch structure. For example, the exterior casing 15HB having a glove shape includes bellows structures 152 having stretchability at portions corresponding to the bases of respective fingers. When the exterior casing 15HB includes the bellows structures 152, the length of each finger of the exterior casing 15HB can be adjusted to fit the size of the hand H. The exterior casing 15HB can be formed by use of, for example, an inner cotton fabric or a urethane material. The material of the exterior casing 15HB is not necessarily a single material. Portions that need to be deformed by an external force such as portions of the bellows structure 152 or portions where a band 120B to be described later is disposed, and the other portions may be formed by use of different materials.

The exterior casing 15HB includes the band 120B as a sensor fastener at each position overlapping with the first blood flow sensor 12 in the plan view illustrated in FIG. 9. At least a part of the band 120B may be fixed (adhered) to the exterior casing 15HB by any means. The fixing length of the band 120B can be adjusted. For example, as illustrated in FIG. 11, the band 120B may be a band provided with a hook-and-loop fastener such as a magic tape (trade name). As illustrated in FIG. 11, the fixing length of the band 120B may be adjusted by sticking together a first surface 1200 constituting one hook-and-loop fastener and the other hook-and-loop fastener provided on at least a part of a second surface 1201 at an arbitrary position. By adjusting the fixing length of the band 120B, the first blood flow sensor 12 can be fixed with respect to the first measurement portion. By using the band 120B, the first blood flow sensor 12 can be fixed with respect to the first measurement portion at a substantially constant contact pressure.

### Third Variation

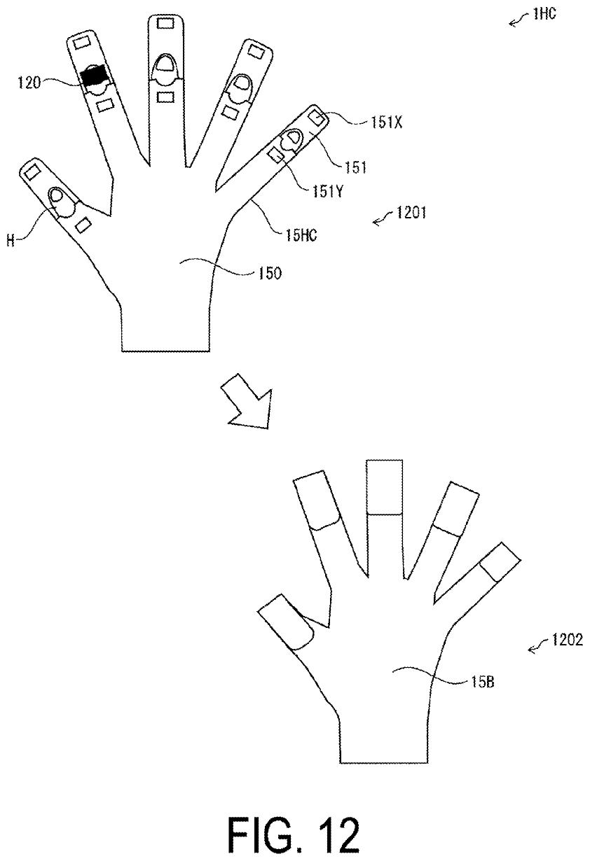

In a third variation, still another variation of the temperature adjustment device 1H according to the first embodiment will be described with reference to FIG. 12.

FIG. 12 is a plan view of an exemplary temperature adjustment device 1HC as a variation of the temperature adjustment device 1H. The plan view illustrated in FIG. 12 is a plan view in which the temperature adjustment device 1HC whose cooling target site is the right hand of a subject is viewed from the back side of the hand.

In the third variation, the temperature adjustment device 1HC includes an exterior casing 15HC, and the Peltier element 11 can be fixed inside the exterior casing 15HC. As illustrated in FIG. 12, the first blood flow sensor 12 is fixed with respect to the first measurement portion by the sensor fastener 120.

The exterior casing 15HC has a glove shape having a five-branch structure. The exterior casing 15HC includes a body 150 covering the palm, the back of the hand, and the respective fingers excluding tip portions thereof, and blades 151. Each of the blades 151 has a shape obtained by extending at least a part of a fingertip portion of the body 150. The reference sign 1201 in FIG. 12 indicates the exterior casing 15HC whose fingertip portions are opened. The fingertip portions of the exterior casing 15HC can be opened as indicated by the reference sign 1201 in FIG. 12. As indicated by the reference sign 1201 in FIG. 12, the tip of each finger can be housed in the exterior casing 15HC by bonding together one bonding means 151X provided at the tip of each of the blades 151 and the other bonding means 151Y provided on the body 150 side. The reference sign 1202 in FIG. 12 indicates the exterior casing 15HC whose blades 151 are closed.

Since the exterior casing 15HC has a configuration in which the fingertip portions can be opened, the hand H may be first inserted into the exterior casing 15HC with the fingertip portions thereof opened, the first blood flow sensor 12 may be fixed to the tip of each finger by the sensor fastener 120, and then the blades 151 may be closed. When the first blood flow sensor 12 is inserted into the exterior casing 15HC after being fixed to the tip of each finger by the sensor fastener 120, the possibility of shifting of the fixing position of the first blood flow sensor 12 can be reduced. Since the first measurement portion can be easily opened, the fixed state of the first blood flow sensor 12 can be easily checked.

### Second Embodiment

Another embodiment of the present disclosure will be described below. For convenience of description, a member having the same function as that of a member described in the embodiments described above is denoted by the same reference sign, and description thereof will not be repeated.

### Pressure Device

A pressure device 500A of the present disclosure is a device that can be used to perform a compression treatment as a therapeutic and preventive strategy for CIPN. In other words, the pressure device 500A of the present disclosure is a device that compresses a target site or the upstream of the target site (a side closer to the heart) in order to perform the above-described compression treatment.

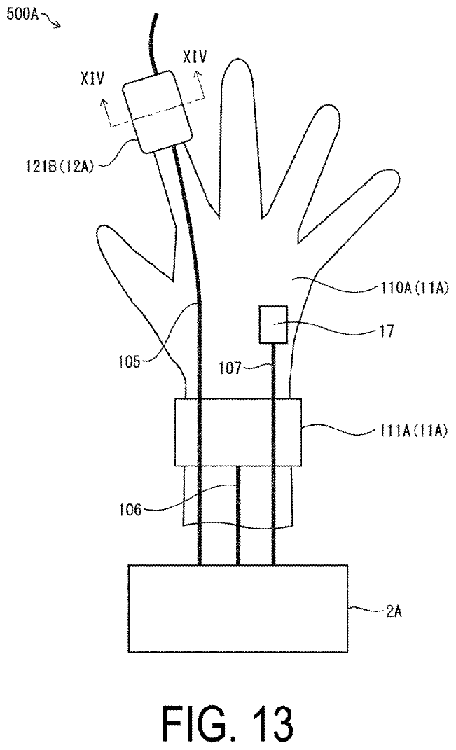

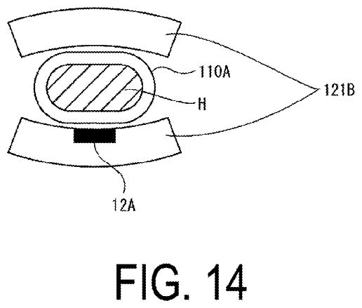

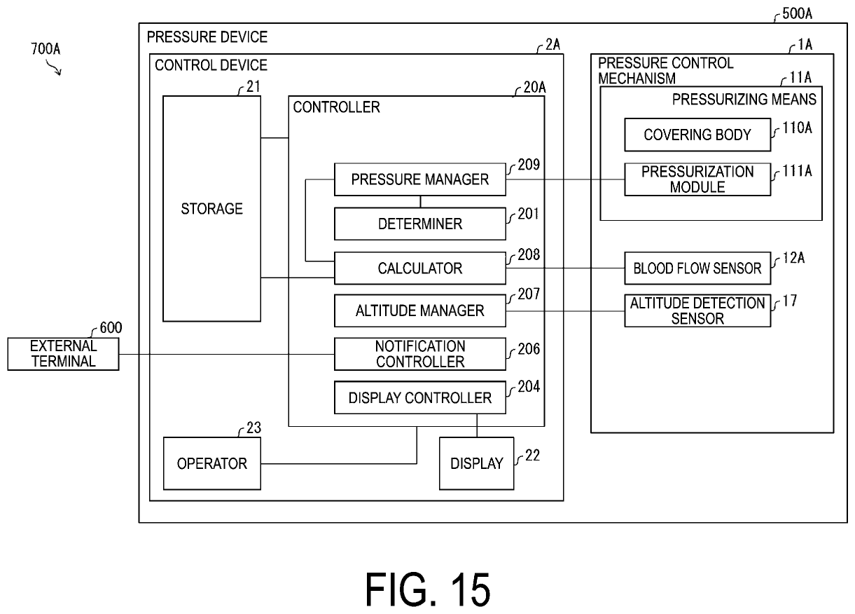

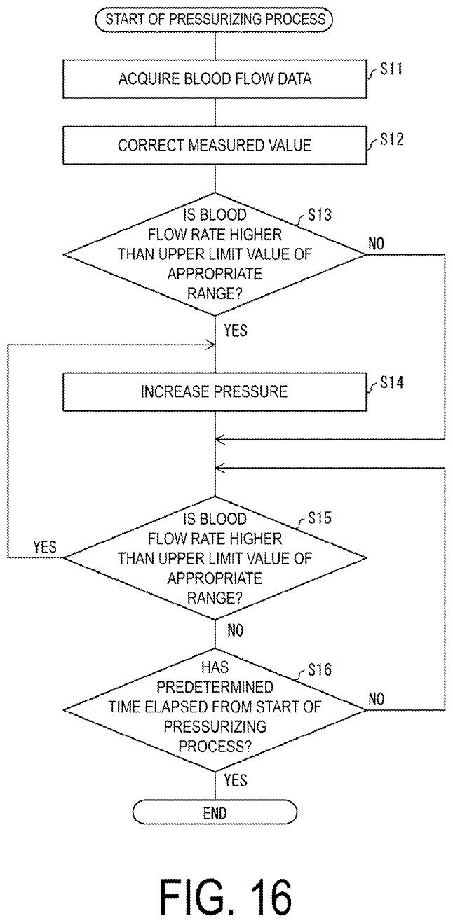

FIG. 13 is a schematic view illustrating an outline of the pressure device 500A according to the second embodiment. FIG. 14 is a cross-sectional view taken along line XIV-XIV indicated by arrows in FIG. 13. FIG. 15 is a functional block diagram illustrating a configuration example of a pressure management system 700A. FIG. 16 is a flowchart illustrating an example of a flow of a blood flow management process by the pressure device 500A.

As illustrated in FIG. 15, the pressure management system 700A includes the pressure device 500A and the external terminal 600 that makes a notification based on information from the pressure device 500A. The pressure device 500A is an example of the blood flow management device according to the present disclosure. The pressure management system 700A is an example of the blood flow management system according to the present disclosure.

As illustrated in FIGS. 13 to 15, the pressure device 500A includes at least one pressure control mechanism 1A, and a control device 2A. As illustrated in FIG. 1, the pressure control mechanism 1A includes, for example, a pressurizing means 11A that adjusts the blood flow in a target site in accordance with blood flow data, a blood flow sensor 12A, and the altitude detection sensor 17. The pressurizing means 11A is an example of a blood flow adjustment means according to the present disclosure.

The pressure control mechanism 1A can be applied to each target site. As an example, FIG. 13 illustrates the pressure control mechanism 1A in which the right hand of a subject is the target site. Although only one pressure control mechanism 1A is illustrated in FIG. 13, a plurality of pressure control mechanisms may be connected to the control device 2A. For example, when both hands are target sites, two pressure control mechanisms 1A can be connected to the control device 2A. When both hands and both feet are target sites, four pressure control mechanisms 1A can be connected to the control device 2A. Hereinafter, the configuration of the pressure control mechanism 1A will be described in detail. However, those skilled in the art can easily understand that another pressure control mechanism 1A in which a site other than the right hand is the target site can have the same and/or similar configuration by appropriately changing the configuration or the shape of the pressurizing means 11A.

The pressurizing means 11A includes, for example, an elastic glove 110A (covering body) and a compression band 111. The pressurizing means 11A does not necessarily need to include both the elastic glove 110A and the compression band 111. The pressurizing means 11A may be achieved only by the elastic glove 110A or only by the compression band 111.

The elastic glove 110A is an example of a covering body according to the present disclosure. The covering body according to the present disclosure is a member that can cover and compress the target site. The covering body reduces the blood flow in the target site by compressing the target site. The covering body according to the present disclosure may have any configuration as long as measurement by the blood flow sensor 12A is possible when the blood flow sensor 12A is attached to the target site via the covering body. The elastic glove 110A may be, for example, a commercially available latex glove. The degree of compression by the elastic glove 110A may be changed by increasing or decreasing the number of gloves or by changing the size of the glove. When the target site is a foot, the covering body may be an elastic sock.

The compression band 111 is a member that is composed of an exterior band having a strip or tubular shape and can be attached to a part of a living body. The compression band 111 is located on the upstream of the target site and reduces the blood flow to the target site by compression. Although FIG. 13 illustrates an example in which the compression band 111 is attached to the wrist, the compression band 111 may be attached to the upper arm. A plurality of compression bands 111 may be attached between the target site and the heart. The degree of compression by the compression band 111 may be changed by changing the tightness of the compression band 111.

The compression band 111 may be a pressurization module 111A. The pressurization module 111A may include, for example, a pressure bag that can expand or contract depending on a voltage applied. When the compression band 111 is the pressurization module 111A, the compression band 111 can be connected to the control device 2A by a connection cable 106. Thus, the degree of compression by the pressurization module 111A can be controlled by the control device 2A.

The blood flow sensor 12A is a sensor that can acquire blood flow data at a measurement portion of the target site. The blood flow sensor 12A can be connected to the control device 2A by a connection cable 105. The blood flow sensor 12A may be an optical sensor that measures a flow rate of a fluid in the measurement portion. The flow rate of the fluid measured by the optical sensor is an example of the blood flow data according to the present disclosure, and is referred to as a primary blood flow rate.

More specifically, the blood flow sensor 12A may include a light emitting element, a light receiving element, and a flow rate calculator. That is, the blood flow sensor 12A may be the flow rate calculation device using the Doppler effect of light described in details in the first embodiment. The light emitting element is an element that can emit light to the measurement portion. The light receiving element is an element that can receive scattered light out of the light emitted to the measurement portion. The flow rate calculator calculates the flow rate of the fluid in the measurement portion based on the frequency component of the scattered light. The flow rate calculation device as the blood flow sensor 12A can output the flow rate of the fluid in the measurement portion to the control device 2A as the primary blood flow rate. According to this configuration, the flow rate of the fluid in the measurement portion can be measured using the optical sensor including the light emitting element and the light receiving element. A controller 20A of the control device 2A may have the function of the flow rate calculator.

As illustrated in FIG. 14, the blood flow sensor 12A may be attached to, for example, an inner side of a pinch clamp 121B that can pinch a tip of a finger. The blood flow sensor 12A may be arranged in direct contact with the measurement portion of the target site. For example, when the elastic glove 110A has a hole corresponding to the measurement portion, the blood flow sensor 12A can be arranged in direct contact with the measurement portion. In this case, the processing by the controller 20A can be simplified.

The blood flow sensor 12A may be arranged in indirect contact with the measurement portion of the target site via the elastic glove 110A. Even when the blood flow sensor 12A is arranged in indirect contact with the measurement portion, the blood flow data can be measured with high accuracy.

The altitude detection sensor 17 may be detachably attached to an outer surface of the elastic glove 110A. The mounting position of the altitude detection sensor 17 is not limited to the example of FIG. 1. The attachment position of the altitude detection sensor 17 may be any position as long as the altitude detection sensor 17 can move in conjunction with the target site when the target site moves with the movement of the body. For example, when the target site is a hand H, the altitude detection sensor 17 may be fixed to an outer surface of the pinch clamp 121B. The altitude detection sensor 17 may be fixed to a part of the hand, the wrist, or the like by a pinch clamp (not illustrated) that can have a configuration same as and/or similar to the configuration of the pinch clamp 121B. The altitude detection sensor 17 can be connected to the control device by a connection cable 107.

The compression band 111, the blood flow sensor 12A, and the altitude detection sensor 17 may be connected to the control device 2A by wire or wirelessly.

The external terminal 600 can make a notification to prompt the user to make a confirmation based on information from the pressure device 500A.

### Control Device

The control device 2A will be described with reference to FIG. 15. The control device 2A includes the controller 20A, the storage 21, the display 22, and the operator 23. The controller 20A includes the display controller 204, the notification controller 206, the altitude manager 207, a calculator 208, a pressure manager 209, and the determiner 201.

The storage 21 may store data necessary for correcting the primary blood flow rate using a correction value related to the light transmitted through the elastic glove 110A. For example, the storage 21 may store a light attenuation rate by the elastic glove 110A as the correction value. The light attenuation rate by the elastic glove 110A is a rate at which the light to be received by the blood flow sensor 12A is attenuated by transmitting through the elastic glove 110A.

The light attenuation rate by the elastic glove 110A can be identified, for example, as follows. First, the following (i) and (ii) are measured. (i) A flow rate of a fluid in a measurement portion measured in a state where the user does not wear the elastic glove 110A (bare hand state). (ii) A flow rate of the fluid in the measurement portion measured via a sheet made of the same material as the elastic glove 110A.

The light attenuation rate by the elastic glove 110A can be determined based on the values (i) and (ii) described above.

Since the light attenuation rate varies depending on the type of elastic glove 110A and can be measured in advance, a model number of the elastic glove 110A and the light attenuation rate thereof may be stored in association with each other in the storage 21. With this configuration, when the user inputs the model number of the glove to be used, the calculator 208 to be described later can acquire the light attenuation rate of the elastic glove 110A to be used as an appropriate correction value.

The calculator 208 calculates the blood flow rate in the measurement portion by correcting the primary blood flow rate using the correction value related to the light transmitted through the elastic glove 110A. For example, the calculator 208 may calculate the blood flow rate in the measurement portion by dividing the primary blood flow rate by the light attenuation rate of the elastic glove 110A. When the pressure device 500A includes the calculator 208, the blood flow rate in the measurement portion can be calculated more accurately even when the measurement is performed via the elastic glove 110A. The calculator 208 may be included in the blood flow sensor 12A.

The display controller 204 may cause the display 22 to display data related to blood flow data. For example, the display controller 204 may cause the display 22 to display the blood flow rate in the measurement portion calculated by the calculator 208. The user can check the indication on the display 22 and adjust the blood flow rate through the control of the degree of compression of the hand, for example, by increasing or decreasing the number of elastic gloves 110A. The blood flow rate in the target site may be adjusted by adjusting the tightness of the compression band 111. The blood flow rate in the target site may be adjusted by changing the positional relationship between the target site and the heart, more specifically, by changing the height of the target site (hand or foot) with respect to the heart.

The pressure manager 209 controls, among pressurizing means 11A, the pressurizing means 11A that is connected to and controllable by the control device 2A. For example, the pressure manager 209 controls the operation of a pressure bag of the compression band 111 including the pressure bag that can expand or contract.

The determiner 201 can determine whether or not the blood flow rate obtained from the blood flow data acquired by the blood flow sensor 12A is within an appropriate range. The appropriate range is an appropriate range of a blood flow rate arbitrarily defined depending on the target site.

In the present embodiment, the pressure device 500A (blood flow management device) manages the blood flow in the target site. The pressure device 500A includes: the blood flow sensor 12A that is arranged in direct or indirect contact with a measurement portion of a target site and can acquire blood flow data being data related to blood flow; and a pressurizing means 11A (blood flow adjustment means) that adjusts the blood flow in the target site in accordance with the blood flow data.

With this configuration, the degree of compression at the target site can be grasped based on the blood flow data. In addition, since the blood flow sensor 12A is arranged in direct or indirect contact with the measurement portion, the blood flow data can be measured with high accuracy and thereby the blood flow can be adjusted.

In the pressure device 500A according to the present embodiment, the target site is a hand or a foot, the pressurizing means 11A includes the elastic glove 110A (covering body) that compresses the hand or the foot, and the blood flow sensor 12A is in contact with the target site via the elastic glove 110A.

With this configuration, the blood flow can be reduced by using the elastic glove 110A. The blood flow sensor 12A is in contact with the target site via the elastic glove 110A, and thereby can measure the blood flow data with high accuracy.

### Pressurizing Process 1

An example of the pressurizing process by the controller 20A will be described using FIG. 16. FIG. 16 is a flowchart illustrating an example of a flow of the pressurizing process by the pressure device 500A according to the second embodiment. This flowchart is a flowchart for a case where the pressurizing means 11A includes, as the compression band 111, the pressurization module 111A including a pressure bag that can expand or contract.

As illustrated in FIG. 16, when the pressure device 500A starts the pressurizing process, the calculator 208 acquires a primary blood flow rate from the blood flow sensor 12A (S11). The calculator 208 corrects the primary blood flow rate acquired in the S11 using the correction value related to the light transmitted through the elastic glove 110A (S12). For example, the calculator 208 calculates the blood flow rate in the measurement portion of the hand H by dividing the blood flow rate acquired in S11 by data of the light attenuation rate of the elastic glove 110A stored in the storage 21. Thus, even when the measurement is performed via the elastic glove 110A, the blood flow rate in the measurement portion can be calculated with the same or similar degree of accuracy as in the case where the blood flow sensor 12A is arranged in direct contact. In other words, even when the blood flow sensor 12A is arranged in indirect contact via the elastic glove 110A, the possibility of the reduction in calculation accuracy of the blood flow rate in the measurement portion can be significantly reduced.

The determiner 201 determines whether or not the blood flow rate calculated in S12 is higher than the upper limit value of the appropriate range (S13). When the determiner 201 determines that the blood flow rate calculated in S12 is higher than the upper limit value of the appropriate range (S13: YES), the pressure manager 209 controls the voltage applied to the pressurization module 111A to increase a compression intensity (pressure) (S14).

When the determiner 201 determines that the blood flow rate calculated in S12 is not higher than the upper limit value of the appropriate range (S13: NO), a pressure intensity is maintained, and the processing proceeds to the next determination (S15).

The determiner 201 determines whether or not the blood flow rate calculated again in a flow same as and/or similar to S11 to S12 is higher than the upper limit value of the appropriate range (S15). When the determiner 201 determines that the blood flow rate is higher than the upper limit value of the appropriate range (S15: YES), the pressure manager 209 controls the voltage applied to the compression band 111 to increase the pressure intensity (returns to the processing in S14). When the determiner 201 determines that the blood flow rate is not higher than the upper limit value of the appropriate range (S15: NO), the pressure intensity is maintained, and the processing proceeds to the next determination (S16).

The controller 20A determines whether or not a predetermined time has elapsed from the start of the pressurizing process (S16). When the controller 20A determines that the predetermined time has elapsed (S16: YES), the controller 20A ends the pressurizing process. When the controller 20A determines that the predetermined time has not elapsed (S16: NO), the controller 20A returns to the processing in S15.

By the controller 20A performing the pressurizing process 1, pressure management can be performed such that the blood flow rate in the measurement portion does not become higher than a threshold value. Consequently, the pharmaceutical exposure in the target site can be reduced, thereby facilitating the management of the pressure therapy that reduces the occurrence of CIPN.

In the pressure device 500A according to the present embodiment, the pressurizing means 11A includes the pressurization module 111A, and the controller 20A can control the pressurization module 111A based on the blood flow rate calculated by the calculator 208.

With this configuration, the blood flow rate in the target site can be controlled by controlling the pressurization module 111A.

### Variation

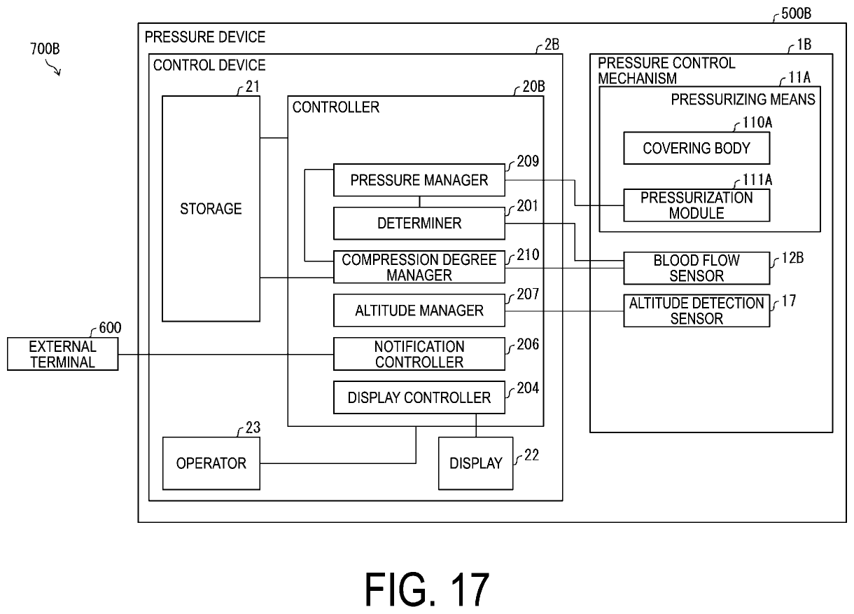

A pressure device 500B as a variation of the pressure device 500A according to the second embodiment, and a pressure management system 700B including the pressure device 500B will be described using FIG. 17. For convenience of description, a member having the same function as that of a member described in the embodiments described above is denoted by the same reference sign, and description thereof will not be repeated.

FIG. 17 is a functional block diagram illustrating a variation of a configuration of the pressure management system 700B according to the second embodiment.

The pressure management system 700B includes the pressure device 500B and the external terminal 600 that makes a notification based on information from the pressure device 500B. The pressure device 500B is an example of the blood flow management device according to the present disclosure. The pressure management system 700B is an example of the blood flow management system according to the present disclosure.

The pressure device 500B includes at least one pressure control mechanism 1B, and a control device 2B. The pressure control mechanism 1B includes a blood flow sensor 12B in substitution for the blood flow sensor 12A included in the pressure control mechanism 1A. The blood flow sensor 12B may include a light emitting element, a light receiving element, and a power spectrum generator. That is, the blood flow sensor 12B may be the flow rate calculation device using the Doppler effect of light described in details in the first embodiment. The light emitting element is an element that can emit light to the measurement portion. The light receiving element is an element that can receive scattered light out of the light emitted to the measurement portion. The power spectrum generator derives a power spectrum indicating a relationship between power and frequency based on the frequency component of the scattered light. The flow rate calculation device as the blood flow sensor 12A can output a power spectrum in the measurement portion to the control device 2A. The power spectrum output by the flow rate calculation device is an example of the blood flow data according to the present disclosure. The power spectrum pulsates in accordance with the pulsation of the blood flow.

The control device 2B includes a controller 20B, the storage 21, the display 22, and the operator 23. The controller 20B includes a compression degree manager 210 in substitution for the calculator 208 included in the controller 20A.

The compression degree manager 210 generates compression degree information indicating the degree of compression at the measurement portion based on the power spectrum. The compression degree information may be, for example, any one of information indicating an area value corresponding to a value obtained by integrating the power spectrum at each time point, information indicating the degree of pulsation of the power spectrum, and information indicating a total sum of distances from an origin to the power spectrum at each frequency. The compression degree information may also be information obtained by converting the information indicating the area value, the information indicating the degree of pulsation, or the information indicating the total sum of the distances from the origin into an index with which the user easily recognizes the degree of compression.

The determiner 201 can determine whether or not the compression degree information generated by the compression degree manager 210 is within an appropriate range. The appropriate range is an appropriate range of information indicating the degree of compression, which is arbitrarily defined depending on the type of information indicating the degree of compression. The compression degree information higher than the appropriate range indicates that the target site is excessively compressed.

The display controller 204 may cause the display 22 to display data related to blood flow data. For example, the display controller 204 may cause the display 22 to display the compression degree information generated by the compression degree manager 210. The display controller 204 may cause the display 22 to display the power spectrum acquired from the blood flow sensor 12B on a real-time basis. The user can check the indication on the display 22 and adjust the blood flow rate, for example, through the control of the degree of compression of the hand by increasing or decreasing the number of elastic gloves 110A. The blood flow rate in the target site may be adjusted by adjusting the tightness of the compression band 111. The blood flow rate in the target site may be adjusted by changing the positional relationship between the target site and the heart, more specifically, by changing the height of the target site (hand or foot) with respect to the heart.

The storage 21 may store reference data necessary for generating the compression degree information in advance.

In the present embodiment, the pressure device 500B may further include the compression degree manager 210. The blood flow sensor 12B includes a light emitting element that can emit light to the measurement portion, a light receiving element that can receive scattered light out of the light, and a power spectrum generator that derives a power spectrum indicating the relationship between power and frequency based on the frequency components of the scattered light. The compression degree manager 210 generates the compression degree information indicating the degree of compression in the measurement portion based on the power spectrum.

With this configuration, information indicating the degree of compression at the measurement portion can be acquired based on the measurement result of the measurement portion using the blood flow sensor 12B.

### Pressurizing Process 2

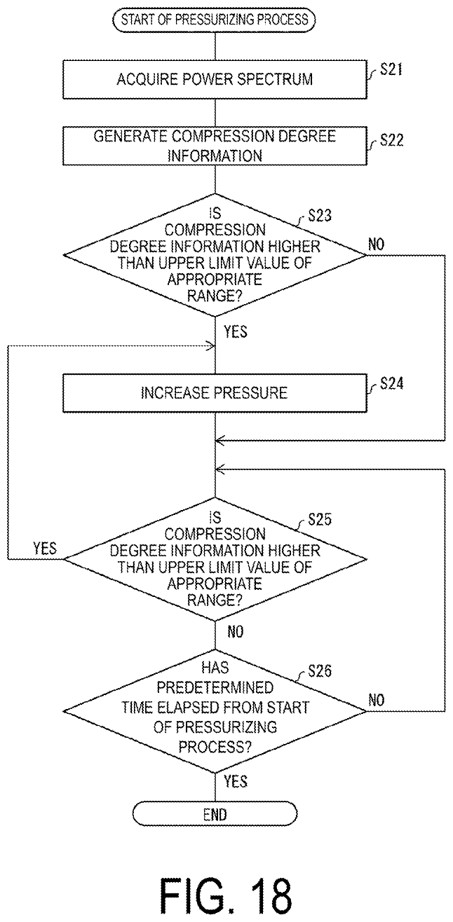

An example of the pressurizing process by the controller 20B will be described using FIG. 18. FIG. 18 is a flowchart illustrating an example of a flow of the pressurizing process by the pressure device 500B according to the second embodiment. This flowchart is a flowchart for a case where the pressurizing means 11A includes, as the compression band 111, the pressurization module 111A including a pressure bag that can expand or contract.

As illustrated in FIG. 18, when the pressure device 500B starts the pressurizing process, the compression degree manager 210 acquires a power spectrum from the blood flow sensor 12B (S21). The compression degree manager 210 generates compression degree information indicating the degree of compression at the measurement portion based on the power spectrum acquired in S21 (S22).

The determiner 201 determines whether or not the compression degree information generated in S22 is higher than an upper limit value of an appropriate range (S23). When the determiner 201 determines that the compression degree information generated in S22 is higher than the upper limit value of the appropriate range (S23: YES), the pressure manager 209 controls the voltage applied to the compression band 111 to increase a compression intensity (S24). When the determiner 201 determines that the compression degree information generated in S22 is not higher than the upper limit value of the appropriate range (S23: NO), a pressure intensity is maintained, and the processing proceeds to the next determination (S25).

The determiner 201 determines whether or not the compression degree information generated again in a flow same as and/or similar to S21 to S22 is higher than the upper limit value of the appropriate range (S25). When the determiner 201 determines that the compression degree information generated in S25 is higher than the upper limit value of the appropriate range (S25: YES), the pressure manager 209 controls the voltage applied to the compression band 111 to increase the pressure intensity (returns to the processing in S24). When the determiner 201 determines that the compression degree information generated in S25 is not higher than the upper limit value of the appropriate range (S25: NO), the pressure intensity is maintained, and the processing proceeds to the next determination (S26).

The controller 20B determines whether or not a predetermined time has elapsed from the start of the pressurizing process (S26). When the controller 20B determines that the predetermined time has elapsed (S26: YES), the controller 20B ends the pressurizing process. When the controller 20B determines that the predetermined time has not elapsed (S26: NO), the controller 20B returns to the processing in S25.

By the controller 20B performing the pressurizing process 2, pressure management can be performed such that the compression degree information does not become higher than the upper limit value of the appropriate range. Consequently, the pharmaceutical exposure in the target site can be reduced, thereby facilitating the management of the pressure therapy that reduces the occurrence of CIPN.

In the pressure device 500B according to the present embodiment, the pressurizing means 11A includes the pressurization module 111A, and the controller 20B can control the pressurization module 111A based on the compression degree information generated by the compression degree manager 210.

With this configuration, the blood flow rate in the target site can be controlled by controlling the pressurization module 111A.

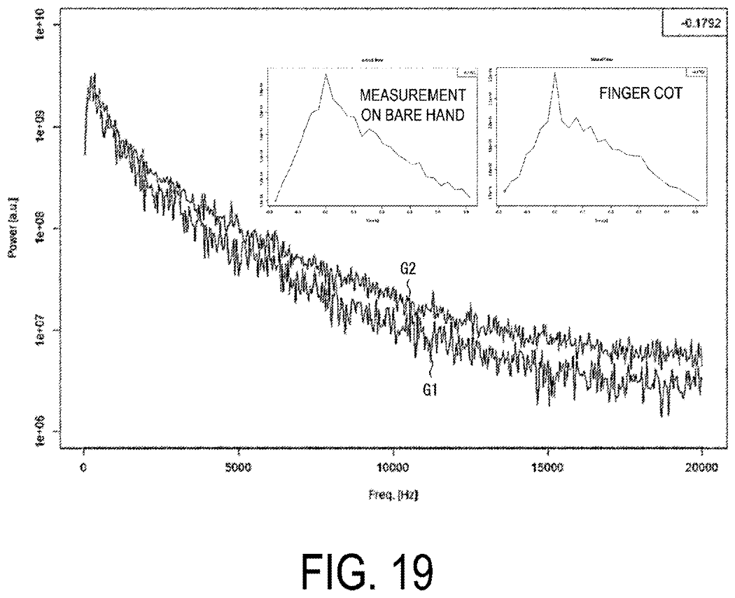

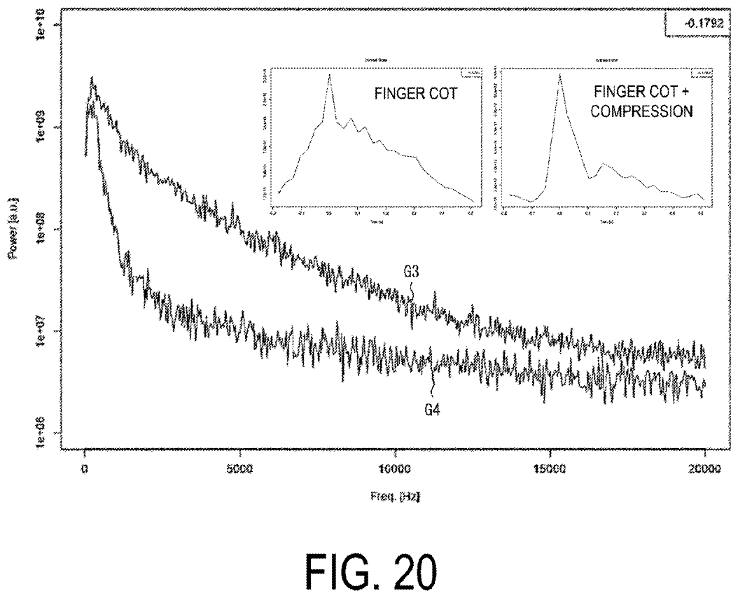

The power spectrum output from the blood flow sensor 12B will be described below with reference to FIGS. 19 and 20.

FIG. 19 is a diagram illustrating a power spectrum measured by the blood flow sensor 12B when the hand is not compressed. FIG. 20 is a diagram illustrating a power spectrum measured by the blood flow sensor 12B when the hand is compressed. The vertical axes in FIGS. 19 and 20 represent power. The horizontal axes in FIGS. 19 and 20 represent frequency.

The reference sign G1 in FIG. 19 indicates a power spectrum in a bare hand state. The reference sign G2 in FIG. 19 indicates a power spectrum in a state where a finger cot (for example, a part of the elastic glove 110A) is attached to the hand. The finger cot has an effect of attenuating light, but does not have an effect of compressing the target site.

The reference sign G3 in FIG. 20 indicates a power spectrum in a state where the finger cot is attached to the hand, as in the case of the reference sign G2. The reference sign G4 in FIG. 20 indicates a power spectrum in a state where the finger cot is attached to the hand, and the hand is compressed.

As illustrated in FIG. 19, the power spectrum indicated by the reference sign G1 and the power spectrum indicated by the reference sign G2 have substantially the same patterns. In other words, the power values at respective frequencies are substantially the same as the values indicated by the reference sign G1 and the reference sign G2. This shows that, even when a member attenuating light such as the finger cot is interposed, the influence on the pattern of the power spectrum is very small. The reason why the power spectrum is less likely to be affected by light attenuation is considered to be that the blood flow sensor 12B calculates the value of the power spectrum by the first moment (flow rate equivalent value) of the power spectrum. The blood flow sensor 12A described above takes into account a light intensity when calculating the flow rate of the fluid based on the frequency components of the scattered light received. On the other hand, the power spectrum output by the blood flow sensor 12B mainly reflect frequency information, and thus is less likely to be affected by the light intensity. Thus, even in the case of the measurement via a covering body, no correction using a correction value related to light is needed.

As illustrated in FIG. 20, the power value at each frequency indicated by the reference sign G3 is larger than the power value at each frequency indicated by the reference sign G4. This shows that the pattern of the power spectrum varies depending on the degree of compression. That is, the compression degree information indicating the degree of compression at the measurement portion can be generated by using the power spectrum.

When the power spectrum of the measurement portion can be viewed on the display 22 on a real-time basis, the degree of compression is ideally adjusted to such an extent that the pulsation of the power spectrum disappears.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

500 Cooling device (blood flow management device)
500A, 500B Pressure device (blood flow management device)
600 External terminal
700 Cooling management system (blood flow management system)
700A, 700B Pressure management system (blood flow management system)
1, 1H, 1HA, 1HB, 1HC Temperature adjustment device
2, 2A, 2B Control device
11 Peltier element (heat transfer module, blood flow adjustment means)
12 First blood flow sensor
12A, 12B Blood flow sensor
120 Sensor fastener
13 Second blood flow sensor
14 Temperature sensor
15, 15H, 15HB, 15HC Exterior casing
16 Cushion member
17 Altitude detection sensor
19 Pressure bag
20, 20A, 20B Controller
110A Elastic glove (covering body, blood flow adjustment means)
111 Compression band (blood flow adjustment means)
111A Pressurization module (blood flow adjustment means)
201 Determiner
202 Temperature manager
203 Calculator
204 Display controller
205 Cooling protocol executor
206 Notification controller
207 Altitude manager
208 Calculator
209 Pressure manager
210 Compression degree manager

1. A device for managing blood flow, comprising:
a blood flow sensor capable of acquiring blood flow data by being arranged in direct or indirect contact with a measurement portion of a target site, the blood flow data being data related to blood flow; and
a blood flow adjustment means for adjusting blood flow in the target site in accordance with the blood flow data.

2. The blood flow management device according to claim 1, wherein
the target site includes at least one of a hand or a foot,
the blood flow adjustment means comprises a covering body configured to compress the hand or the foot, and
the blood flow sensor is in contact with the target site via the covering body.

3. The blood flow management device according to claim 2, further comprising a calculator,
wherein
the blood flow sensor is an optical sensor configured to measure a flow rate of a fluid in the measurement portion, and
the calculator calculates a blood flow rate in the measurement portion by correcting the flow rate using a correction value related to light transmitted through the covering body.

4. The blood flow management device according to claim 3, wherein
the blood flow sensor comprises
a light emitting element capable of emitting light to the measurement portion,
a light receiving element capable of receiving scattered light out of the light emitted, and
a flow rate calculator configured to calculate the flow rate of the fluid in the measurement portion, based on a frequency component of the scattered light.

5. The blood flow management device according to claim 3, further comprising a controller,
wherein
the blood flow adjustment means comprises a pressurization module, and
the controller controls the pressurization module based on the blood flow rate.

6. The blood flow management device according to claim 2, further comprising a compression degree manager,
wherein
the blood flow sensor comprises
a light emitting element capable of emitting light to the measurement portion,
a light receiving element capable of receiving scattered light out of the light emitted, and
a power spectrum generator configured to derive a power spectrum indicating a relationship between power and frequency based on a frequency component of the scattered light,
the compression degree manager generates compression degree information indicating a degree of compression at the measurement portion based on the power spectrum.

7. The blood flow management device according to claim 6, further comprising a controller,
wherein
the blood flow adjustment means comprises a pressurization module,
the controller controls the pressurization module based on the compression degree information.

8. The blood flow management device according to claim 1, further comprising
an altitude detection sensor capable of acquiring altitude data related to an altitude of the measurement portion, and
an altitude manager configured to calculate a displacement in the altitude of the target site from the altitude data.

9. The blood flow management device according to claim 1, wherein
the blood flow adjustment means is at least one first blood flow sensor,
the blood flow adjustment means comprising:
at least one first blood flow sensor capable of acquiring first blood flow data by being arranged in contact with the measurement portion of the target site; and
a controller configured to control the heat transfer module, and
the controller comprises a temperature manager configured to control the heat transfer module based on the first blood flow data.

10. The blood flow management device according to claim 9, further comprising at least one second blood flow sensor capable of acquiring second blood flow data by being arranged at a position different from the first blood flow sensor,
wherein the controller comprises a calculator configured to calculate a blood flow change amount in the target site, based on the first blood flow data and the second blood flow data.

11. The blood flow management device according to claim 9, further comprising
an altitude detection sensor capable of acquiring altitude data related to an altitude of the target site, and
an altitude manager configured to calculate, from the altitude data, a displacement in the altitude of the target site with respect to a cooling start time.

12. A blood flow management system, comprising:
the blood flow management device according to claim 1; and
an external terminal configured to make a notification based on information from the blood flow management device.

13. A device for managing blood flow, the device comprising:
a sensor configured to acquire data including blood flow of a body
a regulator configured to regulate the blood flow; and
at least one processor electrically coupled to the sensor and the regulator, and configured to:
cause the sensor to obtain the data of a measured portion of a target; and
cause the regulator to regulate the blood flow of the target in accordance with the data.

14. The device according to claim 13, further comprising a cover, wherein
the target comprises an arm or a leg of a person,
the sensor is in contact with at least one of the arm or the leg,
the cover is configured to compress the arm or the leg.

15. The device according to claim 14, wherein
the sensor comprises: and
a first element configured to emit light to the targeting in contact with the target;
a second element configured to receive a part of the light scattered from the target, and
the at least one processor is further configured to:
derive a spectrum based on a frequency component of the part of the light, the spectrum indicating a relationship between power and frequency; and
generate information based on the spectrum, the information indicating how strong the cover compresses the arm or the leg.
